(19) United States
(12) Patent Application Publication
(10) Pub. No.: US 2020/0402652 A1
(43) Pub. Date: Dec. 24, 2020

(54) MEDICAL EXAMINATION MANAGEMENT DEVICE, MEDICAL EXAMINATION MANAGEMENT METHOD, AND PROGRAM

(71) Applicant: FUJIFILM Corporation, Tokyo (JP)

(72) Inventor: Yasuhisa KANEKO, Ashigarakami-gun (JP)

(73) Assignee: FUJIFILM Corporation, Tokyo (JP)

(21) Appl. No.: 17/015,092

(22) Filed: Sep. 9, 2020

Related U.S. Application Data
(63) Continuation of application No. PCT/JP2019/010083, filed on Mar. 12, 2019.

(30) Foreign Application Priority Data
Mar. 13, 2018 (JP) 2018-045933

(51) Int. Cl. G16H 40/20; A61B 10/00; A61B 1/31; A61B 10/02; A61B 6/00

(52) U.S. Cl. CPC G16H 40/20; A61B 10/0038; A61B 6/502; A61B 10/0233; A61B 1/31

(57) ABSTRACT

A medical examination management device includes an examination schedule setting unit that acquires examination order information, and sets a schedule of a main examination and a schedule of a simple examination in which a time required from preparation of the examination to obtainment of a result through execution of the examination is shorter than a time in the main examination based on the acquired examination order information, and an examination result acquisition unit that acquires the result of the simple examination. The examination schedule setting unit sets the schedule of the main examination and the schedule of the simple examination such that the result of the simple examination is obtained before the execution of the main examination, and determines that the set schedule of the main examination is cancelable in a case where the result of the simple examination acquired by the examination result acquisition unit is negative.

### CROSS REFERENCE TO RELATED APPLICATION

This is a continuation of International Application No. PCT/JP2019/010083 filed on Mar. 12, 2019, and claims priority from Japanese Patent Application No. 2018-045933 filed on Mar. 13, 2018, the entire disclosures of which are incorporated herein by reference.

### BACKGROUND OF THE INVENTION

### 1. Field of the Invention

The present invention relates to a medical examination management device, a medical examination management method, and a non-transitory computer readable medium storing a program.

### 2. Description of the Related Art

A medical examination support device described in JP2017-054268A specifies an additional examination necessary for confirming a diagnosis based on information regarding examinations performed in the past on a subject and information on findings of a doctor for the subject, and proposes a schedule of the examination. For example, in a case where a determination result of a fecal occult blood examination performed for the subject in the past is positive and the findings of the doctor for the subject are findings suspecting a colorectal cancer, the medical examination support device specifies a colonoscopy examination as an additional examination necessary for confirming the diagnosis, and proposes the schedule of the examination in consideration of vacant schedules of the subject and a medical institution.

### SUMMARY OF THE INVENTION

In general, the fecal occult blood examination is performed as a screening examination in a mass examination, and the colonoscopy examination is performed as a detailed examination for the subject determined to be positive in the screening examination on the day after the screening examination.

Incidentally, the fecal occult blood examination is performed for detecting bleeding from a tumor, but bleeding due to hemorrhoids is detected in reality. Thus, even though the determination of the fecal occult blood examination is positive, a percentage of a negative determination without confirming the tumor being in the colonoscopy examination is about 50% to 70%.

Typically, the colonoscopy examination needs to include many steps such as a colon washing step of causing the subject to excrete excrements by using laxatives, an anesthesia step, an examination step using an endoscope, and a resting step of recovering the subject from anesthesia. A time required for performing the colonoscopy examination is longer than the fecal occult blood examination. Since the endoscope is inserted into the colon, invasiveness is high, and the burden of the subject and the medical institution that executes the examination is large.

In general, in a breast cancer examination, an X-ray mammography examination is executed as a screening examination in a mass examination, and a breast biopsy examination is performed as a detailed examination for the subject for which it is determined to be positive in the screening examination on the day after the screening examination. In the breast biopsy examination, since an aspiration biopsy needle is punctured in the breast, invasiveness is high, and the burden on the subject is large in the same manner as the colonoscopy manner.

It is an object of the present invention to provide a medical examination management device, a medical examination management method, and a non-transitory computer readable medium storing a program capable of reducing burden on a subject and a medical institution that executes an examination.

A medical examination management device according to an aspect of the present invention comprises an examination schedule setting unit that acquires examination order information, and sets a schedule of a main examination and a schedule of a simple examination based on the acquired examination order information, a time required through execution of the simple examination from preparation of the simple examination to obtainment of a result of the simple examination being shorter than a time required through execution of the main examination; and an examination result acquisition unit that acquires the result of the simple examination, wherein the examination schedule setting unit sets the schedule of the main examination and the schedule of the simple examination such that the result of the simple examination is obtained before the execution of the main examination, and determines that the set schedule of the main examination is cancelable in a case where the result of the simple examination acquired by the examination result acquisition unit is negative.

A medical examination management method according to another aspect of the present invention comprises a schedule setting step of acquiring examination order information, and setting a schedule of a main examination and a schedule of a simple examination such that a result of the simple examination is obtained before the execution of the main examination based on the acquired examination order information, a time required through execution of the simple examination from preparation of the simple examination to obtainment of the result of the simple examination being shorter than a time required through execution of the main examination; a simple examination result acquisition step of acquiring the result of the simple examination; and a cancellation step of determining that the set schedule of the main examination is cancelable in a case where the acquired result of the simple examination is negative.

A non-transitory computer-readable recording medium storing a program according to still another aspect of the present invention causes a computer to execute the steps of the medical examination management method.

According to the present invention, it is possible to provide a medical examination management device, a medical examination management method, and a program capable of reducing burden on a subject and a medical institution that executes an examination.

### BRIEF DESCRIPTION OF THE DRAWINGS

FIG. 1 is a block diagram of an example of a medical information system for describing an embodiment of the present invention.

FIG. 2 is a flowchart of an example of a medical examination management method performed by the medical examination management device of FIG. 1.

FIG. 3 is a flowchart of an example of an examination realized by the medical examination management method of FIG. 2.

FIG. 4 is a flowchart of a modification example of the medical examination management method of FIG. 2.

### DESCRIPTION OF THE PREFERRED EMBODIMENTS

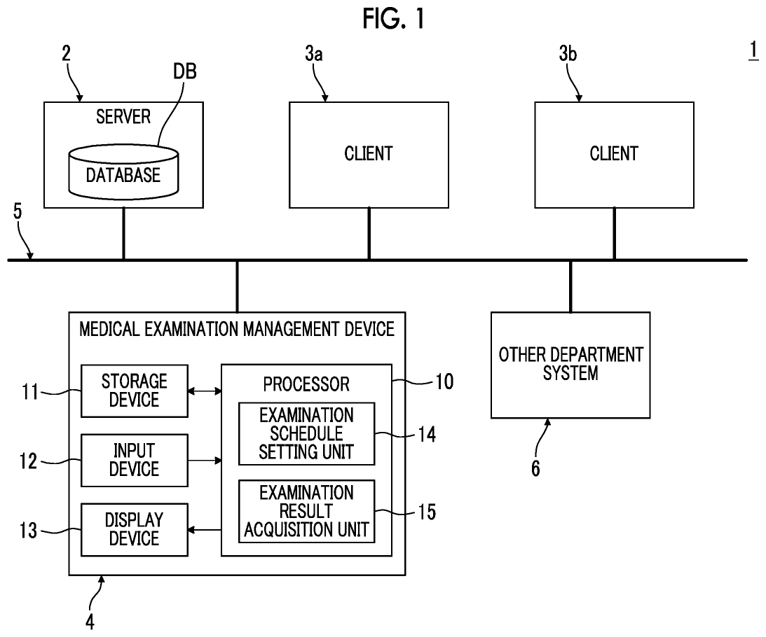

FIG. 1 shows an example of a medical information system for describing an embodiment of the present invention.

A medical information system (hereinafter, simply referred to as a system) 1 is introduced into a medical institution such as a hospital to apply for various examinations executed at the medical institution, set an examination schedule, issue an examination order, and manage an examination result. Hereinafter, a configuration and a function of the system 1 will be described by using a colorectal cancer examination and a breast cancer examination as examples, but a target examination is not limited to the colorectal cancer examination and the breast cancer examination.

The system 1 includes a server 2, a client 3a, a client 3b, and a medical examination management device 4, and the server 2, the client 3a, the client 3b, and the medical examination management device 4 are connected by a network 5 such as a local area network (LAN). The system 1 is also connected to other department system 6 via the network 5. The other department system 6 is, for example, a pathology department system, an endoscope department system, a radiation department system, or an ultrasonic department system.

The server 2 includes a processor such as a central processing unit (CPU), a storage device such as a read only memory (ROM), a random access memory (RAM), and a hard disk drive (HDD), and an input device such as a keyboard and a mouse, and a display device such as a cathode ray tube (CRT) and a liquid crystal display (LCD). A database DB is provided in the storage device of the server 2. The server 2 stores various pieces of information such as the examination application, the examination order, and the examination result in the database DB, and provides the various pieces of information stored in the database DB to the clients 3a and 3b, the medical examination management device 4, and the other department system 6 as necessary.

The client 3a is a computer that includes a processor such as a CPU, a storage device such as a ROM, a RAM, and an HDD, an input device such as a keyboard and a mouse, and a display device such as a CRT and an LCD. The client 3a is installed, for example, at a reception of the medical institution, is operated by a medical clerk, and receives an input of examination application information. The examination application information includes identification information (a name, a date of birth, an age, and a gender) of a subject and an examination type (a colorectal cancer examination or a breast cancer examination). The input examination application information is transmitted from the client 3a to the server 2, and is stored in the database DB.

The client 3b is a computer that includes a processor such as a CPU, a storage device such as a ROM, a RAM, and an HDD, an input device such as a keyboard and a mouse, and a display device such as a CRT and an LCD. The client 3b is installed in, for example, a medical examination room, and is operated by a doctor. The client 3b acquires the examination application information from the database DB, and displays the acquired examination application information on the display device. The displayed examination application information is used by the doctor who conducts a medical examination and explanation of an examination for the subject. The client 3b receives an input of examination order information. The examination order information includes the identification information of the subject, an examination to be ordered, and information indicating whether or not consultation of a primary examination is performed. The input examination order information is transmitted from the client 3b to the server 2, and is stored in the database DB.

The ordered examination is composed of a set of a main examination and a simple examination. A simple examination is an examination in which a time required from preparation of the examination to obtainment of a result through execution of the examination is shorter than a time in a main examination. As an example, in the case of the colorectal cancer examination, the main examination is a colon image examination, and the simple examination is a colon fluid examination. Examples of the colon image examination include a colonoscopy examination and a computed tomography (CT) examination, and examples of the colon fluid examination include a fecal occult blood examination and fecal DNA (deoxyribonucleic acid) examination. As another example, in the case of the breast cancer examination, the main examination is a breast biopsy examination, and the simple examination is a breast image examination. Examples of the breast biopsy examination include a mammography-guided mammotome biopsy examination and an ultrasonic-guided mammotome biopsy examination, and examples of the breast image examination include an X-ray mammography examination and an ultrasonic examination.

The primary examination is a primary examination in a case where the main examination is a secondary examination, and is an already executed examination. In the case of the colorectal cancer examination, the primary examination is, for example, the fecal occult blood examination executed as a screening examination in a mass examination. In the case of the breast cancer examination, the primary examination is, for example, an X-ray mammography examination or a breast ultrasound examination executed as the screening examination in the mass examination.

The medical examination management device 4 is a computer that includes a processor 10 such as a CPU, a storage device 11 such as a ROM, a RAM, and an HDD, an input device 12 such as a keyboard and a mouse, and a display device 13 such as a CRT and an LCD. The processor 10 functions as an examination schedule setting unit 14 and functions as an examination result acquisition unit 15 by operating according to a program stored in the storage device 11 or operating according to a program provided from the server 2 through the network 5.

The examination schedule setting unit 14 acquires the examination order information from the database DB, and sets schedules of the main examination and the simple examination specified by the acquired examination order information. The examination result acquisition unit 15 acquires an examination result of the simple examination specified by the examination order information acquired by the examination schedule setting unit 14 from the database DB.

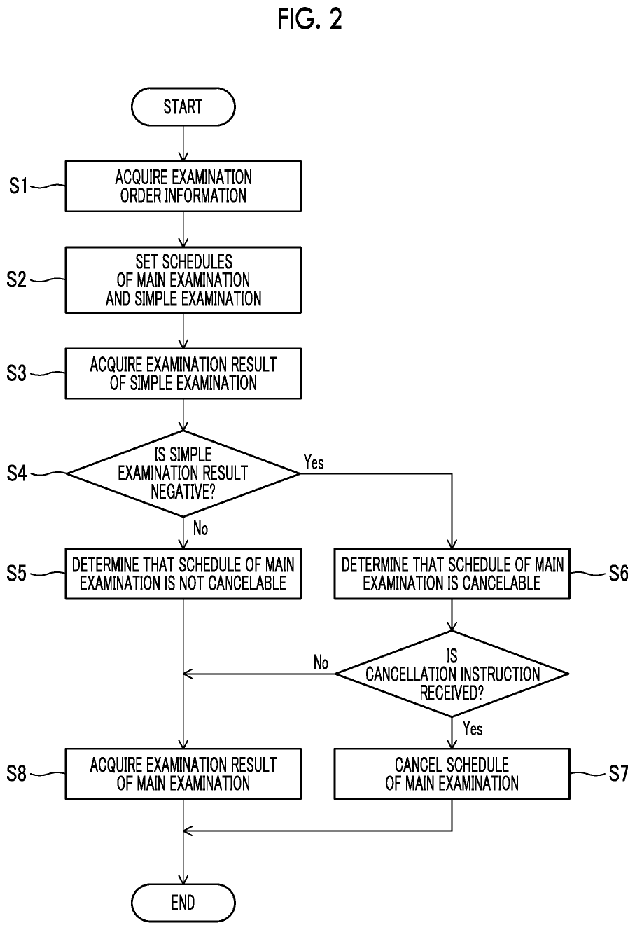

Next, a medical examination management method performed by the medical examination management device 4 will be described. FIG. 2 shows an example of the medical examination management method.

<Schedule Setting Step>

The medical examination management device 4 acquires the examination order information from the database DB (step S1). The examination order information may be automatically transmitted from the server 2 to the medical examination management device 4 in a case where new examination order information is registered in the database DB, or may be transmitted from the server 2 to the medical examination management device 4 by being periodically requested to the server 2 by the medical examination management device 4.

The medical examination management device 4 sets the schedules of the main examination and the simple examination specified by the obtained examination order information (step S2). Here, the simple examination is an examination in which the time required from the preparation of the examination to the obtainment of the result through the execution of the examination is shorter than the time in the main examination, and the examination schedule setting unit 14 sets the schedule of the main examination and the schedule of the simple examination to a schedule in which the examination result of the simple examination is obtained before the execution of the main examination.

The examination order information for which the schedule is set is transmitted to the other department system 6 (the pathology department system, the endoscope department system, the radiation department system, or the ultrasound department system) that executes the main examination and the simple examination via the server 2. In other departments, the main examination and the simple examination will be executed according to the schedules. The examination results of the main examination and the simple examination are transmitted from the other department system 6 to the server 2, and are stored in the database DB.

<Simple Examination Result Acquisition Step>

The medical examination management device 4 acquires the examination result of the simple examination specified by the examination order information acquired in step S1 from the database DB (step S3). The examination result of the simple examination may be automatically transmitted from the server 2 to the medical examination management device 4 in a case where the examination result is registered in the database DB, or may be transmitted from the server 2 to the medical examination management device 4 by being requested to the server 2 by the medical examination management device 4 at a timing at which the examination result of the simple examination is obtained based on the schedule of the simple examination set in step S2 as necessary.

<Cancellation Step>

The medical examination management device 4 determines whether or not the schedule of the main examination which is a set with the simple examination is cancelable based on the examination result of the simple examination acquired in step S3 (step S4).

In a case where the examination result of the simple examination is positive (step S4—No), the medical examination management device 4 determines that the schedule of the main examination is not cancelable, and maintains the schedule set in step S2 (step S5). In this case, the main examination is executed according to the schedule in other departments.

Meanwhile, in a case where the examination result of the simple examination is negative (step S4—Yes), the medical examination management device 4 determines that the schedule of the main examination is cancelable (step S6). The main examination of which the schedule is determined to be cancelable is displayed on, for example, the display device 13 of the medical examination management device 4 and/or the display device of the client 3b, and is notified to the doctor before the execution of the main examination.

Thereafter, the doctor notifies the subject of the examination result of the simple examination, and the intent of the subject is confirmed as to whether or not the subject wants to receive the main examination. In a case where the subject does not want to receive the main examination, an instruction to cancel the schedule of the main examination is input to the client 3b, and the cancellation instruction input to the client 3b is transmitted to the medical examination management device 4 via the server 2.

When the cancellation instruction is received at a timing before the main examination is executed, the medical examination management device 4 cancels the schedule of the main examination based on the schedule of the main examination (step S7). The cancellation of the schedule of the main examination is transmitted to the other department system 6 which executes the main examination via the server 2. Accordingly, the examination is ended without performing the main examination.

In a case where the examination result of the simple examination is negative but the subject wants to receive the main examination or in a case where the doctor recommends to have the examination for a different viewpoint and the subject accepts the main examination according to the recommendation of the doctor, an instruction to continue the main examination (an instruction not to cancel the schedule of the main examination) is input to the client 3b. The continuation instruction input to the client 3b is transmitted to the medical examination management device 4 via the server 2, and the medical examination management device 4 maintains the schedule set in step S2. In this case, the main examination is executed according to the schedule in other departments.

The examination result of the executed main examination is registered in the database DB, and the medical examination management device 4 acquires the examination result of the main examination from the database DB (step S8). The examination result of the main examination is displayed on, for example, the display device of the client 3b, and is transmitted from the doctor to the subject.

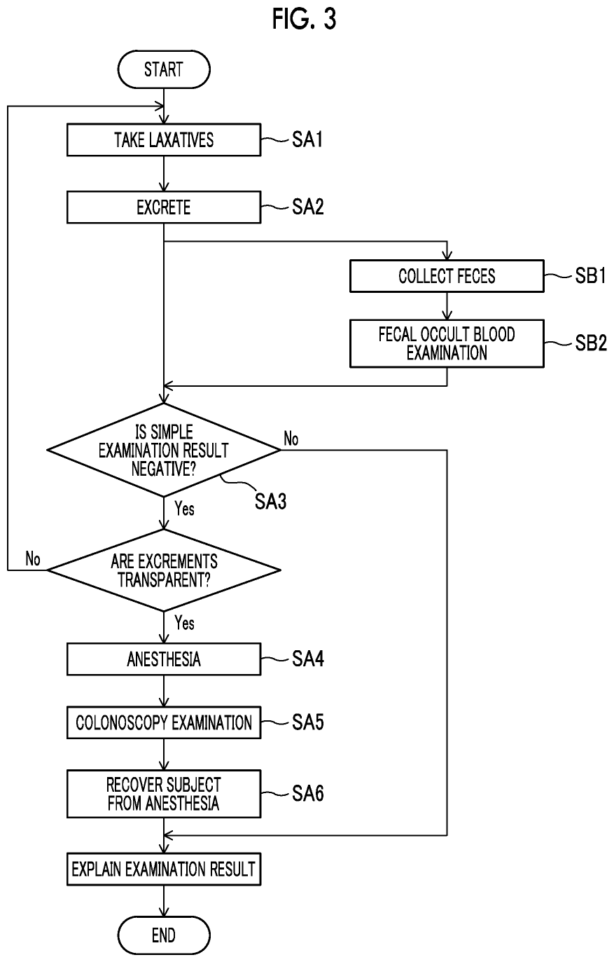

FIG. 3 shows a flow of the examinations realized by the medical examination management method. The examination is the colorectal cancer examination, the main examination is a colonoscopy examination, and the simple examination is the fecal occult blood examination.

The colonoscopy examination which is the main examination includes a step of washing a colon of the subject, a step of anesthetizing the subject, a step of performing the examination by inserting an endoscope into the colon of the subject, and a step of recovering the subject from anesthesia. Meanwhile, the fecal occult blood examination which is the simple examination includes a step of collecting feces from the subject and a step of performing the examination by placing the collected feces in a fecal occult blood examination device.

<Colon Washing Step>

First, the colon of the subject is washed in preparation for the main examination. The subject takes laxatives (step SA1), and excretes excrements (step SA2). Thus, the colon is normally washed. The colon is washed until the excrements become transparent, and typically, the laxatives and the excretion are repeated about 10 times. A time required for washing the colon is typically about 2 hours.

<Feces Collection Step>

The feces collection of the simple examination can be performed together with the washing of the colon, and for example, the feces during a first excretion of the laxative are collected (step SB1).

<Fecal Occult Blood Examination Step>

The feces collected in step SB1 are immediately placed in the fecal occult blood examination device, and are examined (step SB2). A time required from the feces collection to the obtainment of the inspection result through the execution of the fecal occult blood examination is typically less than 2 hours, and is shorter than a time required to wash the colon in preparation for the main examination. Thus, the examination result of the fecal occult blood examination is obtained within a period in which the washing of the colon is continued.

In a case where the examination result of the fecal occult blood examination is positive (step SA3—Yes), the washing of the colon is continued until the excrements become transparent, and after the excrements become transparent, that is, after the washing of the colon is completed, the colonoscopy examination is executed.

<Anesthesia Step, Endoscopic Examination Step, and Anesthesia Recovery Step>

In the colonoscopy examination, the subject is first anesthetized (step SA4), and the endoscope is inserted into the colon of the subject (step SA5). The endoscope is removed from the colon of the subject, and the subject is anesthetized (step SA6).

Meanwhile, in a case where the examination result of the fecal occult blood examination is negative (step SA3—No), the medical examination management device 4 determines that the schedule of the main examination is cancelable as described above. In a case where the schedule of the main examination is determined to be cancelable, as described above, the intent of the subject is confirmed as to whether or not the subject wants to receive the main examination, and in a case where the subject does not want to receive the main examination, the doctor cancels the schedule of the main examination (see steps S6 and S7 of FIG. 2). In a case where the schedule of the main examination is canceled, the examination is ended without performing the anesthesia step of step SA4, the endoscopic examination step of step SA5, and the anesthesia recovery step of step SA6.

As stated above, in a case where the result of the simple examination is negative, it is possible to reduce burden on the subject and the medical institution by canceling the main examination before being executed. Even though the result of the simple examination is negative, it is possible to execute the main examination for the subject who wants the main examination without canceling the main examination, and it is possible to flexibly cope with the examination.

Although the case of the colorectal cancer examination has been described, the burden on the subject and the medical institution can be reduced also in the case of the breast cancer examination. The flow of the breast cancer examination realized by the above-described medical examination management method will be described on the assumption that the main examination is the breast biopsy examination and the simple examination is the breast image examination.

The breast biopsy examination which is the main examination includes a step of imaging the breast of the subject, a step of anesthetizing the subject, a step of performing the examination by puncturing the breast of the subject with an aspiration biopsy needle, and a step of recovering the subject from anesthesia. Meanwhile, the breast image examination which is the simple examination includes a step of imaging the breast of the subject.

<Imaging Step>

First, the breast of the subject is imaged by radiation or ultrasonic waves in preparation for the main examination. This imaging is imaging for calculating puncture position information of the aspiration biopsy needle, and a time required for imaging is typically about 15 minutes. The imaging step of the simple examination can be performed together with the imaging step of the main examination, and one or more images captured in the imaging step of the main examination are used as the captured image of the simple examination.

<Anesthesia Step, Biopsy Examination Step, and Anesthesia Recovery Step>

In a case where the result of the simple examination is positive, the subject is first anesthetized, and the breast of the subject is punctured with the biopsy needle. The biopsy needle is removed from the breast of the subject, and the subject is anesthetized. Subsequently, cells collected by the puncturing are sliced, fixed, stained, and microscopically observed in a pathological examination. Thus, it is determined whether or not there is a tumor.

Meanwhile, in a case where the examination result of the simple examination is negative, the medical examination management device 4 determines that the schedule of the main examination is cancelable as described above. In a case where the schedule of the main examination is determined to be cancelable, as described above, the intent of the subject is confirmed as to whether or not the subject wants to receive the main examination, and in a case where the subject does not want to receive the main examination, the doctor cancels the schedule of the main examination (see steps S6 and S7 of FIG. 2). In a case where the schedule of the main examination is canceled, the examination is ended without performing the anesthesia step, the biopsy examination step, and the anesthesia recovery step.

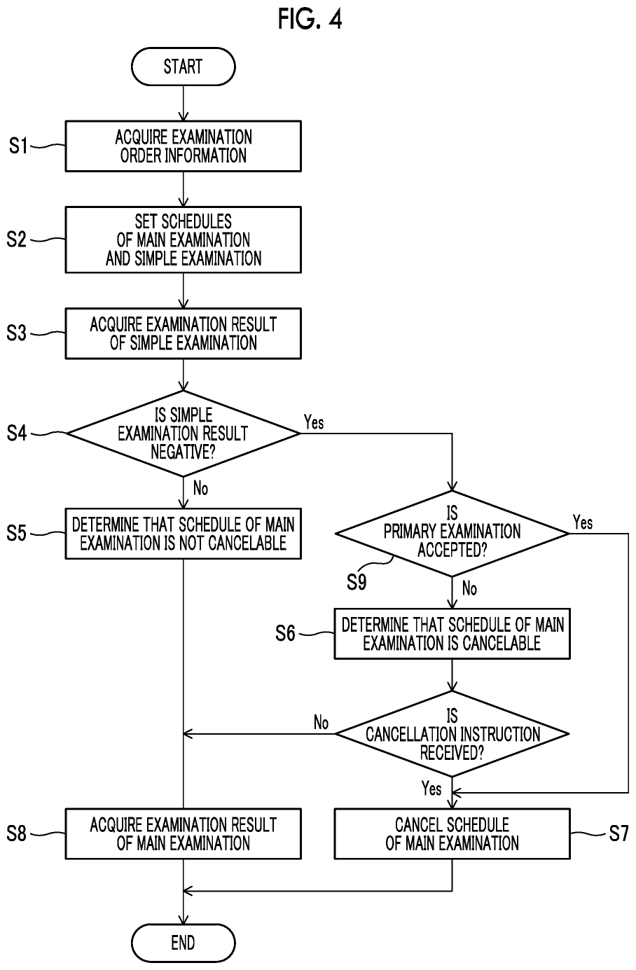

FIG. 4 shows a modification example of the medical examination management method shown in FIG. 2.

In the cancellation step, in a case where the examination result of the simple examination is negative (step S4—Yes) and in a case where the primary examination included in the examination order information is accepted (step S9—Yes), the medical examination management device 4 may cancel the main examination without receiving the cancellation instruction.

Since the result of the primary examination is positive, it is considered that the subject who accepts the primary examination (screening examination in the mass examination) basically accepts the main examination as the secondary examination, and In a case where the result of the simple examination is negative, the main examination can be canceled. Thus, in a case where the medical examination management device 4 cancels the main examination without receiving the cancellation instruction, it is possible to omit the confirmation of the intents of the subject and the doctor, and it is possible to further reduce the burden on the medical institution.

As described above, the medical examination management device disclosed in the present specification comprises an examination schedule setting unit that acquires examination order information, and sets a schedule of a main examination and a schedule of a simple examination in which a time required from preparation of the examination to obtainment of a result through execution of the examination is shorter than a time in the main examination based on the acquired examination order information, and an examination result acquisition unit that acquires the result of the simple examination. The examination schedule setting unit sets the schedule of the main examination and the schedule of the simple examination to a schedule in which the result of the simple examination is obtained before the execution of the main examination, and determines that the set schedule of the main examination is cancelable in a case where the result of the simple examination acquired by the examination result acquisition unit is negative.

In the medical examination management device disclosed in the present specification, the examination schedule setting unit cancels the schedule of the main examination in a case where an instruction to cancel the schedule of the main examination determined to be cancelable is received.

In the medical examination management device disclosed in the present specification, the examination order information includes information indicating whether or not a primary examination is accepted in a case where the main examination is a secondary examination, and the examination schedule setting unit cancels the set schedule of the main examination in a case where the result of the simple examination acquired by the examination result acquisition unit is negative and the primary examination is accepted.

In the medical examination management device disclosed in the present specification, the main examination is a colon image examination, and the simple examination is a colon fluid examination.

In the medical examination management device disclosed in the present specification, the colon image examination is a colonoscopy examination, and the colon fluid examination is a fecal occult blood examination.

In the medical examination management device disclosed in the present specification, the colonoscopy examination includes a step of washing a colon of a subject by causing the subject to excrete excrements, a step of anesthetizing the subject, a step of performing the examination by inserting an endoscope into the colon of the subject, and a step of recovering the subject from anesthesia, and the fecal occult blood examination includes a step of storing feces excreted from the subject in a feces collection container, and a step of performing the examination by placing the feces collection container in a fecal occult blood examination device.

In the medical examination management device disclosed in the present specification, the main examination is a breast biopsy examination, and the simple examination is a breast image examination.

In the medical examination management device disclosed in the present specification, the breast biopsy examination includes a step of imaging a breast of the subject, a step of anesthetizing the subject, a step of puncturing the breast of the subject with a biopsy needle, and a step of recovering the subject from anesthesia, and the breast image examination includes a step of imaging the breast of the subject.

A medical examination management method disclosed in the present specification comprises a schedule setting step of acquiring examination order information, and setting a schedule of a main examination and a schedule of a simple examination in which a time required from preparation of the examination to obtainment of a result through execution of the examination is shorter than a time in the main examination to a schedule in which the result of the simple examination is obtained before the execution of the main examination based on the acquired examination order information, a simple examination result acquisition step of acquiring the result of the simple examination, and a cancellation step of determining that the set schedule of the main examination is cancelable in a case where the acquired result of the simple examination is negative.

In the medical examination management method disclosed in the present specification, in the cancellation step, the schedule of the main examination is canceled in a case where an instruction to cancel the schedule of the main examination is received.

In the medical examination management method disclosed in the present specification, the examination order information includes information indicating whether or not a primary examination is accepted in a case where the main examination is a secondary examination, and in the cancellation step, the set schedule of the main examination is canceled in a case where the result of the simple examination is negative and the primary examination is accepted.

A program disclosed in the present specification causes a computer to execute the steps of the medical examination management method.

The program can be provided by being recorded in a non-transitory computer-readable medium. Such “computer-readable recording medium” includes, for example, an optical medium such as a compact disc ROM (CD-ROM) and a magnetic recording medium such as a memory card. Such a program can be provided by being downloaded via a network.

The present invention can be used for managing various examinations performed in medical institutions.

Although the embodiment of the present invention has been described in detail, the embodiment is merely an example, and the present invention can be implemented in various modification manners without departing from the gist of the present invention. This application is based on the Japanese patent application filed on Mar. 13, 2018 (Japanese Patent Application No. 2018-045933), the contents of which are incorporated herein by reference.

### EXPLANATION OF REFERENCES

1: medical information system
2: server
3a, 3b: client
4: medical examination management device
5: network
6: other department system
10: processor
11: storage device
12: input device
13: display device
14: examination schedule setting unit
15: examination result acquisition unit
DB: database

What is claimed is:

1. A medical examination management device comprising:
an examination schedule setting unit that acquires examination order information, and sets a schedule of a main examination and a schedule of a simple examination based on the acquired examination order information, a time required through execution of the simple examination from preparation of the simple examination to obtainment of a result of the simple examination being shorter than a time required through execution of the main examination; and
an examination result acquisition unit that acquires the result of the simple examination,
wherein the examination schedule setting unit sets the schedule of the main examination and the schedule of the simple examination such that the result of the simple examination is obtained before the execution of the main examination, and determines that the set schedule of the main examination is cancelable in a case where the result of the simple examination acquired by the examination result acquisition unit is negative.

2. The medical examination management device according to claim 1,
wherein the examination schedule setting unit sets the schedule of the main examination and the schedule of the simple examination such that the simple examination is executed during the preparation of the main examination.

3. The medical examination management device according to claim 2,
wherein the main examination is a colon image examination, and the preparation of the main examination includes a step of washing a colon of a subject by causing the subject to excrete excrements.

4. The medical examination management device according to claim 2,
wherein the main examination is a breast biopsy examination, and the preparation of the main examination includes a step of imaging the breast of the subject.

5. The medical examination management device according to claim 1,
wherein the examination schedule setting unit cancels the schedule of the main examination in a case where an instruction to cancel the schedule of the main examination determined to be cancelable is received.

6. The medical examination management device according to claim 1,
wherein the examination order information includes information indicating whether or not a primary examination is accepted in a case where the main examination is a secondary examination, and
the examination schedule setting unit cancels the set schedule of the main examination in a case where the result of the simple examination acquired by the examination result acquisition unit is negative and the primary examination is accepted.

7. The medical examination management device according to claim 1,
wherein the main examination is a colon image examination, and the simple examination is a colon fluid examination.

8. The medical examination management device according to claim 7,
wherein the colon image examination is a colonoscopy examination, and the colon fluid examination is a fecal occult blood examination.

9. The medical examination management device according to claim 8,
wherein the colonoscopy examination includes a step of washing a colon of a subject by causing the subject to excrete excrements, a step of anesthetizing the subject, a step of performing the examination by inserting an endoscope into the colon of the subject, and a step of recovering the subject from anesthesia, and
the fecal occult blood examination includes a step of storing feces excreted from the subject in a feces collection container, and a step of performing the examination by placing the feces collection container in a fecal occult blood examination device.

10. The medical examination management device according to claim 1,
wherein the main examination is a breast biopsy examination, and the simple examination is a breast image examination.

11. The medical examination management device according to claim 10,
wherein the breast biopsy examination includes a step of imaging a breast of the subject, a step of anesthetizing the subject, a step of puncturing the breast of the subject with a biopsy needle, and a step of recovering the subject from anesthesia, and
the breast image examination includes a step of imaging the breast of the subject.

12. A medical examination management method comprising:
a schedule setting step of acquiring examination order information, and setting a schedule of a main examination and a schedule of a simple examination such that a result of the simple examination is obtained before the execution of the main examination based on the acquired examination order information, a time required through execution of the simple examination from preparation of the simple examination to obtainment of the result of the simple examination being shorter than a time required through execution of the main examination;
a simple examination result acquisition step of acquiring the result of the simple examination; and
a cancellation step of determining that the set schedule of the main examination is cancelable in a case where the acquired result of the simple examination is negative.

13. The medical examination management method according to claim 12,
wherein, in the schedule setting step, the schedule of the main examination and the schedule of the simple examination are set such that the simple examination is executed during the preparation of the main examination.

14. The medical examination management method according to claim 12,
wherein, in the cancellation step, the schedule of the main examination is canceled in a case where an instruction to cancel the schedule of the main examination is received.

15. The medical examination management method according to claim 12,
wherein the examination order information includes information indicating whether or not a primary examination is accepted in a case where the main examination is a secondary examination, and
in the cancellation step, the set schedule of the main examination is canceled in a case where the result of the simple examination is negative and the primary examination is accepted.

16. A non-transitory computer-readable recording medium storing a program causing a computer to execute the steps of the medical examination management method according to claim 12.
